(19) United States
(12) Patent Application Publication
(10) Pub. No.: US 2017/0095505 A1
(43) Pub. Date: Apr. 6, 2017

(54) COMBINATION OF XENON AND AN ANTIOXIDANT TO CONTROL A PARKINSON'S DISEASE-TYPE NEURODEGENERATIVE DISEASE

(71) Applicants: L'Air Liquide, Société Anonyme pour I'Etude et I'Exploitation des Procédés Georges Claude, Paris (FR); L'Institut du Cerveau et de la Moelle Epiniere, Paris (FR)

(72) Inventors: Jérémie LAVAUR, Paris (FR); Etienne HIRSCH, Versailles (FR); Patrick MICHEL, Paris (FR); Marc LEMAIRE, Paris (FR)

(21) Appl. No.: 15/312,198

(22) PCT Filed: May 12, 2015
(86) PCT No.: PCT/FR2015/051241

§ 371 Date: Nov. 18, 2016

(30) Foreign Application Priority Data
May 21, 2014 (FR) 1454586

(51) Int. Cl. A61K 33/00; A61K 9/00; A61K 31/355

(52) U.S. Cl. CPC A61K 33/00; A61K 31/355; A61K 9/007

(57) ABSTRACT

The invention relates to a drug combination including xenon gas and at least one antioxidant, in liquid or solid form, for use in treating, slowing, or preventing neurological damage following a neurodegenerative disease in a human being. Preferably, the xenon content is 10% to 80% by volume. Also, the antioxidant is preferably vitamin E, a vitamin E analog, or a vitamin E derivative. The neurodegenerative disease is Parkinson's disease or a disease related to Parkinson's disease.

### CROSS REFERENCE TO RELATED APPLICATIONS

This application is a 371 of International PCT Application PCT/FR2015/051241, filed May 12, 2015, which claims priority to French Patent Application No. 1454586, filed May 21, 2014, the entire contents of which are incorporated herein by reference.

### BACKGROUND

The invention relates to a drug composition comprising a combination of xenon gas and an antioxidant, preferably vitamin E or an analog or derivative thereof, in particular Trolox, used for treating, slowing down or preventing neurological degradation, in relation to, or following a neurodegenerative disease, in particular Parkinson's disease.

NMDA (for N-methyl-D-aspartate) receptors/channels are molecular entities of the plasma membrane of neuronal cells. These receptors are the target of glutamate molecules released into the synaptic and extrasynaptic space, glutamate being an excitatory neurotransmitter which provides communication from one nerve cell to another.

Some neurodegenerative diseases, in particular Parkinson's disease, are characterized by the death of dopaminergic neurons of the brainstem and are clinically manifested by a loss of voluntary motor control, shaking, bradykinesia and stiffness.

Neuronal degeneration in Parkinson's disease probably involves excitotoxicity, i.e. a mechanism associated with over-stimulation of NMDA receptors caused by an excess of glutamate neurotransmitter in the extracellular space (Metha et al., Eur. J. Pharmacol, 2013).

Indeed, it has been shown in cell and animal models mimicking Parkinson's disease that a dysfunction of excitatory amino acid transporters (EAATs) carried by glial cells and neurons results in dopaminergic cell death (Nafia et al., J Neurochem, 2008; Swanson et al., Ann Neurol, 2011).

Furthermore, dopaminergic neurons in culture are most particularly vulnerable when they undergo stress caused by exogenous glutamate (Douhou et al., J Neurochem, 2001).

Moreover, after lesion of the dopaminergic system in rats and monkeys, there is an increase in the activity of the subthalamic neurons which are responsible for glutamatergic excitatory innervation of dopaminergic neurons (Wallace et al., Brain, 2007).

In addition, it has been established that dopamine (DA) protects against glutamate-induced toxicity (Vaarmann et al., Cell Death and Disease, 2013). Thus, a DA depletion caused by a dopaminergic lesion could in itself promote dopaminergic neuron vulnerability to excitotoxic stress.

However, there is at the current time no drug which is actually efficacious and satisfactory from a therapeutic point of view and which makes it possible to treat, slow down and/or prevent neurological degradation following a neurodegenerative disease, in particular Parkinson's disease, in a human being.

In view of this, the problem which arises is that of providing a drug which makes it possible to treat, slow down and/or prevent neurological degradation following a neurodegenerative disease, in particular Parkinson's disease, in a human being.

### SUMMARY

The solution according to the present invention is a drug combination comprising xenon gas and at least one antioxidant in liquid or solid form, for use for treating, slowing down or preventing neurological degradation following a neurodegenerative disease in a human being.

In other words, the invention relates to a gas composition, i.e. an inhalable gaseous drug, based on xenon, for use in combination with at least one antioxidant in liquid or solid form, for treating, slowing down or preventing neurological degradation following a neurodegenerative disease in a human being.

As appropriate, the drug combination or composition according to the invention can comprise one or more of the following technical features:
it contains between 10% and 80% by volume of xenon;
the antioxidant is vitamin E, a chemical analog of vitamin E or a chemical derivative of vitamin E;
the antioxidant is Trolox, i.e. 6-hydroxy-2,5,7,8-tetramethylchromane-2-carboxylic acid, which is a water-soluble chemical analog of vitamin E (Davies et al., Biochem J., 1988);
the antioxidant is in liquid form;
the antioxidant is Trolox in liquid form;
the neurodegenerative disease is Parkinson's disease or a disease related to Parkinson's disease, preferably Parkinson's disease;
the xenon gas is administered to the patient before, concomitantly with or after administration of the antioxidant, preferably after administration of the antioxidant;
the neurological degradation is related to an excessive entry of Ca²⁺ ions into one or more populations of vulnerable neurons of the patient or human being to be treated;
the xenon is in a mixture with oxygen and/or nitrogen;
the proportion of oxygen is at least 21% by volume;
it consists of xenon and oxygen or of xenon, nitrogen and oxygen;
the human being is more than 30 years old, preferably at least 40 years old, in particular at least 50 years old;
the proportion of xenon is at least 20% by volume;
the proportion of xenon is at most 75% by volume, preferably the proportion of xenon is at most 60% by volume;
the xenon is administered to the patient by inhalation;
the human being treated or to be treated, i.e. the patient, is a man or a woman;
the xenon is used, i.e. administered, in an effective amount or proportion;
the drug contains a non-anesthetic amount of xenon, i.e. a sub-anesthetic amount;
the xenon is mixed with oxygen before or at the time of its inhalation by the patient, or is in the form of a “ready-to-use” gas mixture as a premix with oxygen, and optionally contains another gas compound, for example nitrogen;
the drug consists of a gas mixture formed from oxygen and nitrogen;
the gas is administered to the patient one or more times per day;
the gas is administered to the patient for an inhalation time of a few minutes to a few hours, typically between 15 minutes and 6 hours, preferentially less than 4 hours;
the duration, the dosage regimen and the frequency of administration of the xenon and/or the antioxidant depend on the progression of the neurological condition of the patient under consideration and these parameters will be preferentially set by the physician or care staff depending on the neurological condition of the patient under consideration;
the xenon (or the gas mixture containing the xenon) is packaged in a gas cylinder having a volume (water equivalent) ranging up to 50 liters, typically of about from 0.5 to 15 liters, and/or at a pressure of less than or equal to 350 bar absolute, typically a pressure of between 2 and 300 bar. Preferably, the gas cylinder is made of steel, aluminum or composite material, and is equipped with a valve or a pressure regulator that is integrated, making it possible to control the flow rate and optionally the pressure of the gas delivered;
during the treatment, the xenon (or the gas mixture containing the xenon) is administered to the patient by inhalation by means of a face mask or nasal mask or of nasal goggles or by means of any other system for administration of an inhalable gas.

More specifically, in the context of the present invention, it has been demonstrated that the combination of the xenon and an antioxidant, in particular Trolox or vitamin E, results in a synergistic action of these compounds and that such a combination can constitute a promising treatment for neurological degradation resulting from neurodegenerative diseases, of Parkinson's disease type.

In particular, good results have been obtained by combining xenon with Trolox or 6-hydroxy-2,5,7,8-tetramethylchromane-2-carboxylic acid, which is a soluble vitamin E analog, which acts by inhibiting oxidative stress and more particularly by inhibiting lipid peroxidation.

Indeed, by making such a combination, the inventors of the present invention have demonstrated a synergistic effect of a combination between xenon and the antioxidant of Trolox type, as detailed and exemplified hereinafter.

Such a Xe/antioxidant combination is based in particular on the modes of action of these types of compounds or molecules.

Thus, xenon has excitatory glutamatergic signaling pathway-inhibiting properties (Dinse et al., Br J Anaesth, 2005), via its antagonistic action on NMDA receptors, but also on α-amino-3-hydroxy-5-methylisoxazole-4-propionate (AMPA) receptors, and also on kainite receptors, which make up glutamate ionotropic receptors.

Moreover, as stated, Trolox acts by inhibiting oxidative stress associated with lipid peroxidation (Guerreiro et al., J Neurochem, 2009).

Consequently, by making a joint combination of xenon and antioxidant, in particular of the type Trolox or vitamin E or a derivative thereof, the inventors have demonstrated a synergy of action resulting from a blocking of glutamatergic receptors by the xenon and an intracellular antioxidant effect of the Trolox, of the vitamin E or of a derivative thereof. In other words, the inventors of the present invention have noted that xenon makes it possible to reveal or reinforce the beneficial effects of the antioxidant through a synergistic effect.

According to another aspect, the invention also relates to a therapeutic treatment method for treating, slowing down or preventing at least one neurological degradation following a neurodegenerative disease in a human patient, i.e. a human being, in which:
i) a human patient suffering from a neurodegenerative disease or liable to be suffering from such a neurodegenerative disease is identified,
ii) a gaseous drug containing xenon is administered to said patient by inhalation, and
iii) at least one antioxidant in liquid or solid form, in particular Trolox, vitamin E or a derivative thereof, is administered to said patient so as to obtain neuronal protection and thus to treat, slow down or prevent at least one neurological degradation resulting from the neurodegenerative disease is said patient.

Preferably, in step i):
the human patient is a man or a woman;
the human patient is more than 30 years old, preferably at least 40 years old, in particular at least 50 years old;
the patient is identified by a physician or the like;
the patient is identified by technical screening examination;
the neurodegenerative disease capable of causing at least one neurological degradation of Parkinson's disease type, preferably Parkinson's disease;
said neurological degradation comprises an excessive entry of Ca²⁺ ions into one or more populations of vulnerable neurons of the patient.

Preferably, in step ii):
the antioxidant is administered in liquid form;
at least one antioxidant is administered enterally, i.e. orally;
Trolox (vitamin E analog), vitamin E or a derivative thereof is administered to the patient as antioxidant;
Trolox in liquid form is administered to the patient;
at least one antioxidant is administered to said patient before, concomitantly with or after inhalation of the xenon by the patient.

Preferably, in step iii):
the duration, the dosage regimen and the frequency of administration of the xenon are chosen and/or set according to the progression of the neurological condition of the patient under consideration;
an effective amount of xenon is administered;
a non-anesthetic amount of xenon is administered;
from 10% to 80% by volume of xenon, preferably between 20% and 50% by volume of xenon, is administered;
the xenon is mixed with oxygen before or at the time of its inhalation by the patient, preferably with at least 21% by volume of oxygen;
a ready-to-use gas mixture consisting of xenon and oxygen (binary mixture) or of xenon, oxygen and nitrogen (ternary mixture) is administered;
the xenon gas is administered to the patient one or more times per day;
the xenon gas is administered to the patient for an inhalation time of a few minutes to a few hours, typically between 15 minutes and 6 hours, preferentially less than 4 hours;
the xenon gas is administered by means of a face mask or nasal mask or of nasal goggles or by means of any other system or device for administration of gas to a patient.

### BRIEF DESCRIPTION OF THE DRAWING

The invention will now be understood more clearly by virtue of the following examples, given by way of illustration but without limitation, and of the appended figure which shows the synergistic protective effects of xenon and Trolox in a cell model mimicking chronic Parkinsonian degeneration.

### DESCRIPTION OF PREFERRED EMBODIMENTS

### EXAMPLES

In order to demonstrate the efficacy of the combination of xenon and an antioxidant according to the present invention, a dopaminergic neuron cell model was set up, in which model the neuronal death is triggered by blocking the glutamate (GLU) reuptake systems by means of a prolonged treatment with L-trans-pyrrolidine-2,4-dicarboxylic acid (PDC), which is a synthetic compound of which the mechanism of action was previously described by Zuiderwijk et al., (Europ J Pharmacol, 1994).

The technique implemented is described below and the results obtained are illustrated in the appended figure.

Protocol for Obtaining Mesencephalon Primary Cultures

Cultures are prepared from mesencephalon of rat embryos, taken from gestating female Wistar rats on day 15.5 of gestation.

The method for obtaining the mesencephalon cultures comprises obtaining a homogeneous cell suspension via mechanical dissociation, i.e. non-enzymatic dissociation, of the embryonic tissue, using Leibovitz L15 medium (Sigma Aldrich).

Aliquots of this suspension are added to Nunc 48-well plates, which were precoated with a thin layer of polyethyleneimine (1 mg/ml, borate buffer, pH 8.3) so as to enable the adhesion of the neuronal cells (cf. Toulorge et al., Faseb J, 2011).

The seeding density is between approximately 80 000 and 100 000 cells/cm².

The mesencephalon cultures are maintained in neurobasal culture medium, containing a B27 cocktail without antioxidant, N2 supplement, glutamine (2 mM) and a penicillin/streptomycin cocktail (cf. Nafia et al., J Neurochem, 2008). The medium and supplements thereof are available from Life Technologies.

Up to the time when the effects of the gases of interest are evaluated, the cultures are placed in a conventional enclosure thermostatically controlled at 37° C., in which the CO₂ is maintained at 5% by volume and wherein the atmosphere is saturated with water.

No change of culture medium is carried out throughout the culture period.

Pharmacological Treatments of the Cultures p The degenerative process is triggered by applying a glutamate reuptake system blocker, L-trans-pyrrolidine-2,4-dicarboxylic acid or PDC (Zuiderwijk et al., Europ J Pharmacol, 1994).

The antioxidant, namely Trolox, is added to the cultures before the application of PDC.

These two products, i.e. PDC and Trolox, are commercially available from the companies RD Systems and Sigma Aldrich, respectively.

Maintenance of the Cultures under a Controlled Gaseous Atmosphere

Once the pharmacological treatments have been carried out, the multiwell plates containing the cells in culture and the plate used to humidify the internal compartment of the chamber are placed on a metal base which receives the Plexiglas incubation chamber. The two parts (base and Plexiglas chamber) are butt-joined together by screwing.

A gas mixture of interest comprising (% by volume): 20% of O₂, 5% of CO₂ and 75% of the gas tested is then injected into the incubation chamber, with open inlet and outlet valves, while at the same time controlling the output flow rate by means of a flow meter.

The gases tested are nitrogen and xenon.

The reference output flow rate, set for air at 10 liters/min, is corrected according to the density of the mixture used.

When the CO₂ measurement reaches 5% at the outlet, the injection of the gas mixture is stopped and the chamber is made totally airtight by closing the inlet and outlet valves.

The exposure chamber is then placed in an enclosure at 37° C. throughout the experimental protocol.

Immunodetection of Tyrosine Hydroxylase (TH) and Cell Counts

After breaking the airtightness by opening the inlet and outlet valves and unscrewing the chamber from its base, the cultures are fixed with 4% formaldehyde in PBS for 12 min and then incubated at 4° C. with an anti-TH monoclonal antibody (dilution 1/5000) for 2 days.

This antibody is revealed with an IR DYE800 anti-mouse second antibody (Euromedex, 1/10 000 in PBS). The image acquisition is carried out with an infrared imager of Odyssey type (Li-COR Biosciences) and the quantification of the neurons is carried out by means of the ICY software (Institut Pasteur).

The results obtained in the model of mesencephalic degeneration by chronic treatment with PDC reveal additive protective effects between the xenon and the Trolox which are summarized in the following table and represented in the figure.

TABLE
Summary of the principal results of the in vitro study
on the mesencephalon cultures
Mesencephalon cultures analyzed on day (D) 16

| | Gas mixture | Survival |
|---|---|---|
| | (20% O₂ + 5% | of TH- |
| | CO₂ + 75% of gas | positive |
| Treatments D 13-D 16 (4 days) | tested); % by vol. | neurons |
| Control group I | N₂ | +++ |
| Control group II | Xe | +++ |
| PDC (100 μM) group | N₂ | − |
| PDC (100 μM) group | Xe | ++ |
| PDC (100 μM) + Trolox (0.1 μM) group | N₂ | − |
| PDC (100 μM) + Trolox (0.1 μM) group | Xe | +++ |
| PDC (100 μM) + Trolox (0.3 μM) group | N₂ | − |
| PDC (100 μM) + Trolox (0.3 μM) group | Xe | +++ |
| PDC (100 μM) + Trolox (1 μM) group | N₂ | + |
| PDC (100 μM) + Trolox (1 μM) group | Xe | +++ |
| PDC (100 μM) + Trolox (10 μM) group | N₂ | ++ |
| PDC (100 μM) + Trolox (10 μM) group | Xe | +++ |

In the above table:
a favorable response, synonymous with a decrease in the level of neuronal cell death in the presence of the treatments of interest, is denoted by a “+”, “++”, or “+++” sign (+++ = level of reference);
conversely, an unfavorable response is represented by a “−” sign, synonymous with an increase in the neuronal death.

Trolox acts as a lipid peroxidation inhibitor and xenon acts by blocking NMDA receptors.

PDC, for its part, induces a degenerative process by preventing the reuptake of the glutamate endogenously produced and released by the neuronal cells in culture, thereby resulting in an overactivation of the neuronal NMDA receptors and in a stimulation of the intracellular production of oxygen radical species.

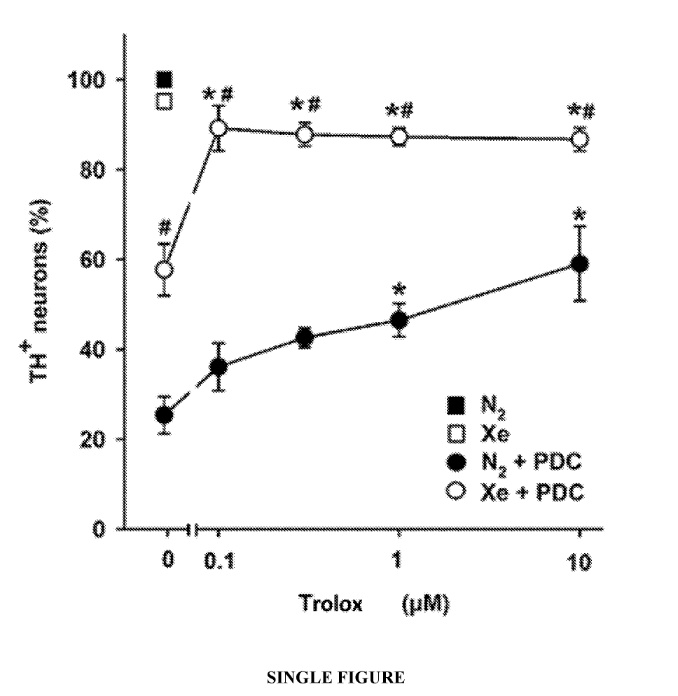

In the light of the results illustrated in the appended FIG. 1, it is noted that the combination of xenon and an antioxidant, namely in this case Trolox, produces a synergistic neuroprotective effect compared with the effect of each molecule taken separately.

In fact, an actual synergy of action of the xenon/Trolox combination is set up, entirely unexpectedly.

First of all, these tests showed that PDC causes a considerable loss of dopaminergic neuronal cells, at a concentration of 100 μM, under an atmosphere containing 75 vol % of nitrogen. However, these deleterious effects of PDC are partially prevented when the nitrogen is replaced with xenon, since the survival rate then goes from 25.4% to 57.7%.

Notably, the Trolox which has no significant effect in this cell model when it is applied alone at 0.1-0.3 μM, under an atmosphere containing 75% of nitrogen, becomes effective in the presence of xenon, namely in this case 75% of xenon, making it possible to achieve a survival rate of about 90% under these conditions.

A potentiation of the effects of the xenon is also observed when Trolox concentrations ≧1 μM are applied, which exert a protective effect when they are applied in 75% of nitrogen.

The results recorded on the figure appended hereto illustrate the synergistic protective effects of xenon and of Trolox in a cell model mimicking chronic Parkinsonian degeneration. These results were obtained on rat mesencephalon cultures which were treated at the end of day 12 in vitro and for the following 4 days, with a glutamate reuptake inhibitor, PDC (100 μM), under an atmosphere containing 75% of nitrogen (N₂ 75) or 75% of xenon (Xe 75), in the presence or absence of Trolox, tested at from 0.1 to 10 μM.

The cultures are then recovered for fixing and analysis. The neuronal survival is quantified under the various experimental conditions tested, by counting the number of cell bodies immunopositive for the dopaminergic marker TH.

The results are expressed in % (±SEM) of the mean values of the cultures not treated with PDC, maintained under 75% of nitrogen (control condition).

Thus, the statistical study carried out by means of a Student's t test, in the case of simple comparisons between two groups, or by one-way analysis of variance (ANOVA), followed by a Dunnett's test for multiple comparisons performed relative to a reference group (n=9 for each experimental point) demonstrates that:
in an atmosphere containing 75% of xenon (compared with 75% of nitrogen in the control condition), an increase in the survival of the dopaminergic neurons treated with PDC is observed whether Trolox is present or absent in the culture medium (# p<0.05, vs corresponding cultures under 75% of nitrogen, exposed to PDC, in the presence or absence of Trolox);
in an atmosphere containing 75% of nitrogen, Trolox produces a significant protective effect starting from 1 μM and in an atmosphere containing 75% of xenon, starting from 0.1 μM (*p<0.05, vs cultures exposed only to 100 μM of PDC in the same gaseous atmosphere).

Therefore, xenon, when it is combined with an antioxidant, in particular of the type Trolox or vitamin E or a derivative thereof, produces a synergistic effect in the treatment, slowing down or prevention of neurological degradation following a neurodegenerative disease of Parkinson type, in particular Parkinson's disease.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A drug combination comprising xenon gas and at least one antioxidant in liquid or solid form, for use for treating or slowing down neurological degradation following a neurodegenerative disease in a human being.

2. The drug combination of claim 1, containing between 10% and 80% by volume of xenon.

3. The drug combination of claim 1, wherein the antioxidant is vitamin E, a vitamin E analog or a vitamin E derivative.

4. The drug combination of claim 1, wherein the antioxidant is Trolox.

5. The drug combination of claim 1, wherein the antioxidant is Trolox in liquid form.

6. The drug combination of claim 1, wherein the neurodegenerative disease is Parkinson's disease or a disease related to Parkinson's disease.

7. The drug combination of claim 1, wherein the xenon gas is mixed with oxygen gas.

8. The drug combination of claim 1, wherein the xenon gas is mixed with at least 21% by volume of oxygen gas.

9. The drug combination of claim 1, wherein the xenon gas is mixed with at least 21% by volume of oxygen gas and nitrogen gas.

10. The drug combination of claim 1, wherein the human being is more than 30 years old.

11. The drug combination of claim 1, wherein the proportion of xenon gas is at least 20% by volume and at most 75% by volume.

12. A drug therapy for treating or slowing down neurological degradation following a neurodegenerative disease in a human being comprising administering to the human being a xenon gas and at least one antioxidant in liquid or solid form to thereby treat or slow down the neurological degeneration.

13. The method of claim 12 wherein the xenon gas is 20%-75% by volume of a gaseous composition administered to the human being by inhalation.

14. The method of claim 13, wherein the gaseous composition further contains at least 21% oxygen gas.

15. The method of claim 13, wherein the antioxidant is Vitamin E or a known Vitamin E derivative or analog known to be used as a functional substitute for Vitamin E.

16. The method of claim 14, wherein the antioxidant is Trolox in liquid form.
